(19) United States
(12) Patent Application Publication
(10) Pub. No.: US 2017/0071505 A1
(43) Pub. Date: Mar. 16, 2017

(54) Dry Powder Delivery System and Method

(71) Applicant: METHOCHOLINE, Ontario (CA)

(72) Inventors: Alex Stenzler, Long Beach, CA (US); Steve Han, Huntington Beach, CA (US)

(21) Appl. No.: 15/267,927

(22) Filed: Sep. 16, 2016

Related U.S. Application Data
(60) Provisional application No. 62/219,214, filed on Sep. 16, 2015.

(51) Int. Cl. A61B 5/091; A61B 5/00; A61M 15/00

(52) U.S. Cl. CPC A61B 5/091; A61M 15/0021; A61M 15/0045; A61M 15/0006; A61M 15/002; A61M 15/0051; A61M 15/0048; A61M 15/0041; A61M 15/0016; A61M 15/0066; A61M 15/0091; A61B 5/742; A61M 2202/064; A61M 2205/3334; A61M 2206/16; A61M 2205/583

(57) ABSTRACT

A dry powder inhalation device includes in certain embodiments a spin chamber and a cartridge for holding multiple capsules. The capsules contain a dry powder that in certain embodiments includes methacholine. In certain embodiments, an adjustable limiting mechanism is configured to limit inhaled volume through the second passageway. In certain embodiments, the cartridge moves linearly or radially though a portion of the device. In certain embodiments, capsules in the cartridge align with a port in the spin chamber to load into the spin chamber. User inhalation on a mouthpiece of the device generates a vortical airflow and causes the capsules to spin within the chamber, releasing a portion of the dry powder for inhalation. In certain embodiments, the device is used for an inhalation diagnostic challenge. The inhalation diagnostic challenge can be automated, using a graphical user interface, an imaging system, user feedback mechanisms, and electromechanical components within the device for automating device operation.

### CROSS-REFERENCE TO RELATED APPLICATIONS

This application claims priority to U.S. provisional application No. 62/219,214 filed on Sep. 16, 2015, incorporated herein by reference in its entirety.

### BACKGROUND OF THE INVENTION

The diagnosis of asthma (reactive airways disease) requires evidence that the airways will narrow in response to a bronchial provocation exposure to a response eliciting agent. These agents may include for example methacholine, mannitol, cold dry air, or exercise. Bronchial provocation is most frequently performed using an aerosolized liquid methacholine chloride formulation, such as the bronchoconstrictor agent Provocholine (Methapharm Inc., Ontario, Canada). Initially, methacholine chloride is typically provided to health care professionals in powder form, and it is later reconstituted into a liquid dilution (using for example sodium chloride) when it is ready for use. During a methacholine challenge test, the diluted methacholine formulation is inhaled by the subject at increasing concentrations. Patients with asthma demonstrate a particular sensitivity that leads to bronchoconstriction as compared to healthy patients. This difference in response is the basis for the inhalation diagnostic challenge.

There are however shelf life problems with this approach. Once the methacholine powder is mixed with the liquid excipient, it must be used in a very short period (ranging in certain cases from a same day to a two week shelf life). In addition, it needs to be prepared by a pharmacist, which limits where it can be used. An additional problem with aerosolized solutions is the amount of droplets that are discharged into the room that could cause bronchoconstriction in people who should not be exposed to the drug. These include testing technicians, technologists, nurses, physicians as well as other patients in the laboratory for testing. In many cases, testing requires a special exposure chamber with filtered air and a droplet containment component. Additionally, aerosol methods require periodic precision calibration of the aerosol delivery device, which is a significant and elaborate procedure. More than 20 million Americans suffer from asthma and require a correct diagnosis through performance of a bronchial provocation test. Since testing can only be performed at a small number of institutions with proper testing resources and facilities, and private practice physicians are rarely are able to perform this test because of the limitations identified, a need for a better system would benefit the general public.

Another problem associated with approaches for bronchial provocation that require deep inspiration is that deep inspiration can promote both bronchial constriction or bronchial dilatation. Therefore, when performing bronchial provocation testing with methods that use a deep inspiration method to total lung capacity, a lower sensitivity to the response eliciting agent may be exhibited when compared to alternative methods using tidal breathing.

Although dry powder agents such as mannitol may be beneficial to circumvent certain issues mentioned above related to liquid forms (e.g. shelf life, preparation, and test facility limitations) in that there are no exhaled particles to contaminate the air in the laboratory facility, current dry powder inhalation approaches are nonetheless problematic because they typically require deep inspirations. Thus, there is a need in the art for a device and method to deliver dosimeter type challenges in a dry powder form while limiting the range of inspiration during the dose delivery.

### SUMMARY OF THE INVENTION

In one embodiment, a dry powder delivery device includes a mouthpiece including a proximal and distal opening with a first passageway extending therebetween; a spin chamber including a port and an ejection door, wherein the spin chamber is connected to the first air passageway; a housing including a second passageway, wherein the second passageway is connected to the first passageway and further comprises an airflow sensor; a controller operably connected to the airflow sensor; and a cartridge including a plurality of dry powder receptacles that are configured to align with the port as the cartridge moves relative to the housing. In one embodiment, the second passageway includes an adjustable limiting mechanism configured to limit inhaled volume through the second passageway. In one embodiment, the adjustable limiting mechanism is configured to limit inhaled volume through the second passageway to a level between 0.1 and 3.0 liters of air. In one embodiment, the adjustable limiting mechanism is configured to limit flow through the second passageway to a level between 30 and 120 liters of air per minute. In one embodiment, the adjustable limiting mechanism is configured to limit flow through the second passageway to a level between 60 and 90 liters of air per minute. In one embodiment, the adjustable limiting mechanism is controlled by a motor operably connected to the controller. In one embodiment, the adjustable limiting mechanism is a valve. In one embodiment, the cartridge is configured to move through the housing in a substantially linear direction. In one embodiment, the cartridge is configured to move through the housing in a substantially radial direction. In one embodiment, the cartridge is moved through the housing by a motor operably connected to the controller. In one embodiment, the cartridge is substantially rectangular. In one embodiment, the cartridge is substantially circular. In one embodiment, the sensor is a pressure transducer. In one embodiment, the mouthpiece is a detachable mouthpiece connected to the housing. In one embodiment, a lattice structure is disposed across at least one of the distal opening and the spin chamber. In one embodiment, a first and second air inlet are connected to the spin chamber and positioned to facilitate vortical airflow when a negative pressure is applied to the mouthpiece. In one embodiment, the housing includes a performance indicator including a plurality of light emitting elements. In one embodiment, the plurality of light emitting elements includes a first and second light emitting elements, each having different colors. In one embodiment, the ejection door is actuated by a motor operably connected to the controller. In one embodiment, the dry powder delivery device further includes a piercing element configured to advance at least partially into the spin chamber. In one embodiment, the dry powder delivery device further includes an imaging system including a first camera trained on the spin chamber and operably connected to the controller. In one embodiment, the imaging system includes a second camera trained on the cartridge and operably connected to the controller. In one embodiment, the second pathway includes a one-way elastomeric valve configured to favor proximally directed inspiratory airflow towards the mouthpiece.

In one embodiment, a method for performing an inhalation diagnostic challenge includes inhaling on a mouthpiece of a device; generating a vortical airflow within a spin chamber of the device to spin a first capsule, the first capsule containing dry powder and disposed within the spin chamber, the dry powder containing a first amount of methacholine; releasing at least a first portion of the dry powder from the first capsule; and inhaling an aerosolized form of the first portion of the dry powder released from the capsule. In one embodiment, the method includes advancing the cartridge in a linear direction through the device. In one embodiment, the method includes advancing the cartridge in a radial direction through the device. In one embodiment, the method includes adjusting an airflow through the device by adjusting a volume limiting mechanism. In one embodiment, the method includes ejecting the capsule from the spin chamber.

In one embodiment, the method includes performing an inhalation diagnostic challenge. The method includes the steps of advancing a cartridge comprising a plurality of dry powder capsules through a device housing to deposit a first dry powder capsule into a spin chamber, inhaling on a mouthpiece of the device to generate a vortical airflow within the spin chamber to spin the first capsule, the first capsule containing dry powder containing a first amount of methacholine, releasing at least a first portion of the dry powder from the first capsule, and inhaling an aerosolized form of the first portion of the dry powder released from the capsule. In one embodiment, the second amount of methacholine is greater than the first amount of methacholine. In one embodiment, the second amount of methacholine is substantially two times greater than the first amount of methacholine. In one embodiment, the second amount of methacholine is substantially four times greater than the first amount of methacholine.

In one embodiment, a method for performing an inhalation diagnostic challenge includes advancing a cartridge comprising a plurality of dry powder receptacles through a device housing to deposit a first amount of dry powder into a spin chamber; inhaling on a mouthpiece of the device to generate a vortical airflow within the spin chamber, the first amount of dry powder containing a first amount of methacholine; and inhaling an aerosolized form of the first amount of dry powder. In one embodiment, the method includes advancing the cartridge through the device to deposit a second amount dry powder into the spin chamber; inhaling on the mouthpiece of the device to generate a vortical airflow within the spin chamber, the second amount of dry powder containing a second amount of methacholine different from the first amount of methacholine; and inhaling an aerosolized form of the second amount of dry powder. In one embodiment, the second amount of methacholine is greater than the first amount of methacholine. In one embodiment, the second amount of methacholine is substantially two times greater than the first amount of methacholine. In one embodiment, the second amount of methacholine is substantially four times greater than the first amount of methacholine.

In one embodiment, a dry powder delivery device includes a mouthpiece including a proximal and distal opening with a first passageway extending therebetween; a spin chamber including a port and an ejection door, wherein the spin chamber is connected to the first air passageway; a housing including a second passageway, wherein the second passageway is connected to the first passageway and further includes an airflow sensor and an adjustable limiting mechanism configured to limit inhaled volume through the second passageway; and a controller operably connected to the airflow sensor and the adjustable limiting mechanism. In one embodiment, the adjustable limiting mechanism is configured to limit inhaled volume through the second passageway to a level between 0.1 and 3.0 liters of air. In one embodiment, the adjustable limiting mechanism is configured to limit flow through the second passageway to a level between 30 and 120 liters of air per minute. In one embodiment, the adjustable limiting mechanism is configured to limit flow through the second passageway to a level between 60 and 90 liters of air per minute. In one embodiment, the adjustable limiting mechanism is controlled by a motor operably connected to the controller. In one embodiment, the adjustable limiting mechanism is a valve. In one embodiment, the dry powder delivery device includes a cartridge including a plurality of dry powder receptacles that are configured to align with the port as the cartridge moves relative to the housing.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a perspective view of a dry powder delivery system having a sliding cartridge according to an exemplary embodiment.

FIG. 2 is a side view of the dry powder cartridge loaded with dry powder capsules shown in FIG. 1.

FIG. 3 is a magnified side view of a capsule loaded in the spin chamber of the dry powder delivery system shown in FIG. 1.

FIGS. 4A and 4B are side views of the dry powder delivery system shown in FIG. 1. FIG. 4A shows the dry powder delivery system with the ejection door closed, and FIG. 4B shows the dry powder delivery system with the election door open.

FIG. 5A is a side view and FIG. 5B is a perspective view of a dry powder delivery system having a spinning cartridge according to an exemplary embodiment.

FIG. 6A is a side view and FIG. 6B is a perspective view of internal components of the dry powder delivery system shown in FIGS. 5A and 5B. FIG. 6C is a perspective view of internal components of the dry powder delivery system shown in FIGS. 5A and 5B with an outer casing and certain components removed.

FIG. 7 is a system diagram of a dry powder delivery system according to an exemplary embodiment.

FIGS. 8A-8M are images of a graphical user interface during an inhalation diagnostic challenge according to an exemplary embodiment.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems and methods for dry powder delivery. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

“FVC” are used herein means forced vital capacity.

Measurement of the effects of the challenge during bronchial provocation testing is usually performed with a forced vital capacity maneuver. However, alternative measurement techniques such as forced oscillation, body plethysmography, airways resistance, electrical impedance tomography, etc. may also be used to determine the effect of the challenge. Throughout this disclosure “FVC” may represent any of the methods known in the art to determine a change in airway or lung function that reflects the effect of the administration of an airway challenge.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein are systems and method for dry powder delivery.

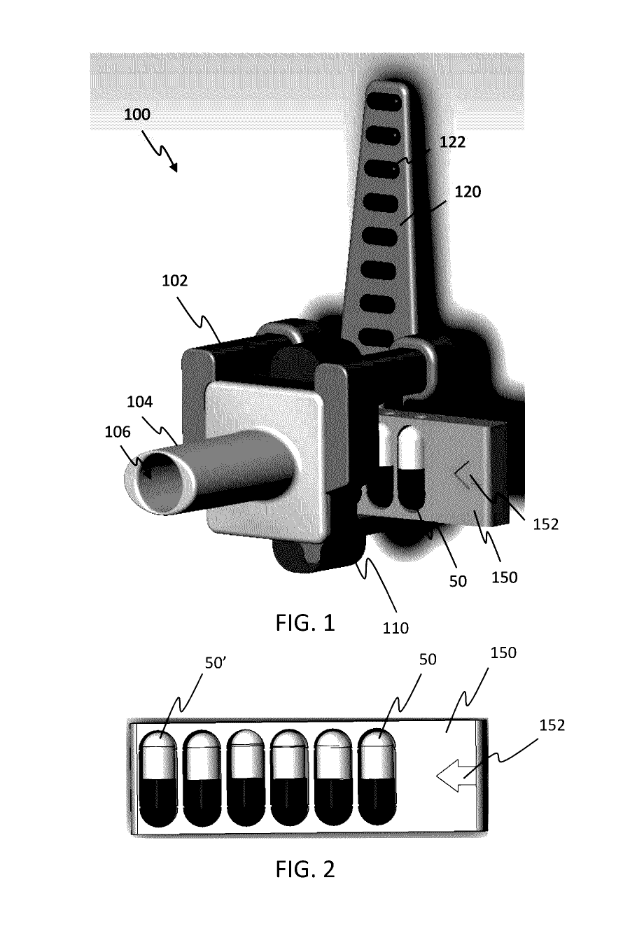

With reference now to the exemplary embodiment shown in FIGS. 1-4B, a dry powder delivery system 100 includes a housing 102 that can accommodate a sliding cartridge 150. The cartridge 150 (shown isolated in FIG. 2) holds multiple dry powder capsules 50 that each sit in individual dry powder capsule receptacles. In one embodiment, the dry powder is in the form of methacholine as previously discussed in U.S. Pat. No. 6,462,090 to Slutsky et al. In one embodiment, the powder is placed directly into the capsule receptacle or other power chamber or reservoir without the need for a capsule. In certain embodiments, the dry powder capsules increment in order to contain a five dose quadrupling dose (doses that quadruple with each increment), while in other embodiments, the dry powder capsules increment in order to contain a ten dose doubling dose (doses that double with each increment) or other volumes to account for the actual volume of powder delivered from the capsule. Embodiments of the invention include capsules that include a single dose, a partial dose, a multi-dose or combinations of increasing, decreasing or variable dosages per capsule. According to certain embodiments, certain capsules, such as the first capsule 50′, is a test dose which may be an empty capsule, or alternatively a capsule containing an excipient (e.g., lactose), a dry powder or another material that does not cause an adverse reaction with the user's airway. In certain embodiment, a multi-dose cartridge includes one training capsule, one excipient capsule, followed by 10 doubling doses. Generally, the capsule 50 is a dry powder storage chamber suitable for use with devices described herein. The capsule 50 can be manufactured from materials including hypromellose (HPMC). The cartridge 150 advances through the housing 102 along a particular line or direction, which may be indicated on the cartridge 150 by a marker, such as an arrow 152. The cartridge 150 is advanced manually by a user or technician, or can advance automatically using an electromechanical mechanism, such as a rack and pinion or actuation advancement mechanism. In certain embodiments, the cartridge 150 is restricted to advancing one capsule at a time to the next adjacent capsule. An imaging system (described in further detail below) can provide various levels of automation, such as determining when advancement to the next dosage is allowed. In certain embodiments, the cartridge can be advanced or pull back over multiple capsules, which can allow for skipping ahead or pulling back to a particular capsule and dosage.

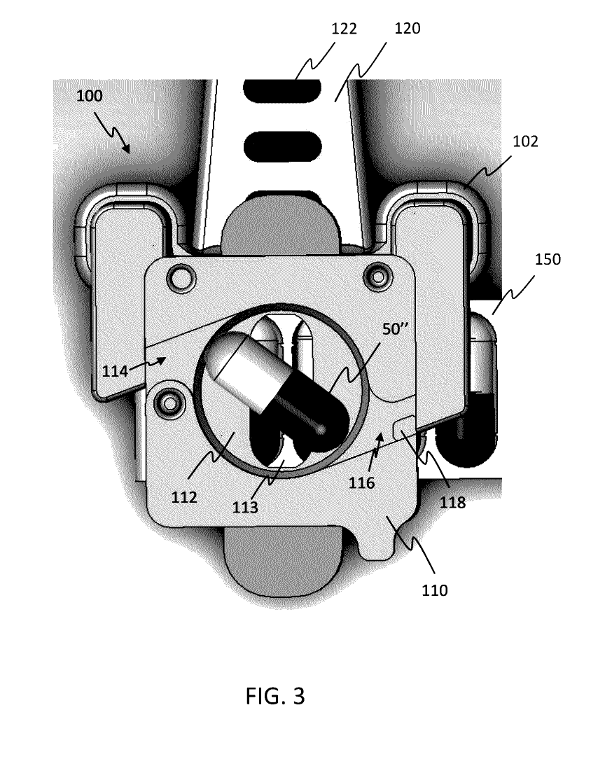

As each capsule 50 is advanced, it is positioned to align with the opening in the spin chamber 112 through a port 113, as shown with more detail in FIG. 3. The capsule 50 is drawn into the spin chamber 112 through the port 113 by the inhalation effort of the test subject. In certain embodiments, one or more pins, such as a dual pin structure is used to puncture the capsule 50 just before it enters the spin chamber 112. Once the capsule 50″ is punctured, the system 100 is ready for a user inhalation. In certain embodiments using powder without a capsule, the alignment of the capsule receptable, chamber or reservoir with the opening in the spin chamber makes the system ready for powder to enter the spin chamber and for user inhalation. The spin chamber 112 is shaped to help the loaded capsule 50″ spin either partially or fully within the spin chamber 112 in response to an airstream from a negative air pressure created by the test subject's inhalation effort. Spin chamber openings 114, 116 are placed in an offset configuration to draw air into the spin chamber 112, creating a vortical airflow that promotes spinning of the loaded capsule 50″. In certain embodiments the flow path into the spin chamber may be from the rear of the capsule receptacle. With reference back to FIG. 1, the mouthpiece 104 protrudes from the housing 102 so that a user can comfortably close their mouth around the outer surface of the mouthpiece 104 and generate a negative air pressure by inhaling. The mouthpiece 104 has a mouthpiece opening 106 or lumen that extends to the spin chamber 112. In certain embodiments, the mouthpiece 104 is a disposable component that can easily be attached by a snap-fit and detached from the housing 102, so that a different mouthpiece 104 can be attached between users.

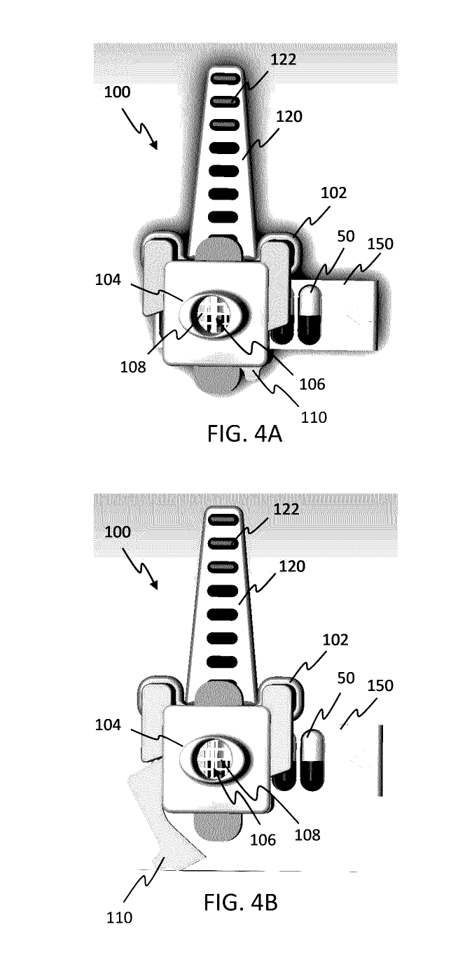

With reference now to FIGS. 4A and 4B, a lattice structure 108 separates the mouthpiece opening 104 and the spin chamber 112. The lattice structure 108 has a number of openings to allow airflow therethrough, deagglomerate the particles and also acts to keep the capsule confined within the spin chamber 112 during user inhalation. In certain embodiments, openings of the lattice structure 108 are angled and offset so that airflow through the lattice structure 108 further promotes a vortical airflow within the spin chamber 112, along with the spin chamber openings 114, 116. As the pierced loaded capsule 50″ spins in response to user inhalation, dry powder enters the airstream and is aerosolized as it travels into the user's airway. After the dosage of powder is cleared from the loaded capsule 50″, an ejection door 110 swings open (see FIG. 4B), and the used capsule is ejected out of the base of the device 100.

In one embodiment, at least one sensor 118 is set in an air pathway and is in communication with the user's airflow moving through the system 100 for measuring inspiratory effort. The sensor, such as a pressure or flow transducer, can be attached in or near the spin chamber 112 or the mouthpiece opening 106, or in an alternate pathway in airflow communication with the mouthpiece 104. In certain embodiments, the sensor 118 is used to provide feedback to the user through a performance indicator 120 that is within view to the user during the challenge. In an exemplary embodiment, the performance indicator 120 includes a number of light emitting elements 122, such as light emitting diodes, that light to reflect user performance. Performance can include, for example, a flow based or pressure based reading from the sensor 118. The light emitting elements 122 can be stacked and color coded to provide additional types of feedback information to the user. In certain embodiment, a flow limiter is also utilized to provide inspiratory flow clamping and limit the airflow through the device to control maximum inspiratory flow. For example, in certain embodiments, different sized mouthpieces 104 with various diameter openings are provided to medical technicians, so that a proper flow limit is established during setup phases of the challenge. In other embodiments, the lattice structure 108 is interchangeable, providing a multitude of cross-sectional opening profiles to allow more or less airflow through the mouthpiece 104. Certain embodiments may also limit airflow by adjusting the size of certain airflow pathways within the system 100, such as by using an iris valve to narrow or opening the spin chamber openings 114, 116.

In certain embodiments, an imaging system can utilize a camera to image capsules, while image processing software run by a controller is used to confirm that correct dosages are loaded into each capsule, and that dry powder is sufficiently cleared from capsules following user inhalation. The image processing system could also be used to verify what percentage of a particular dosage has been inhaled following a user inhalation. In certain embodiments, the imaging system includes a dual camera setup for verifying correct doses are loaded into the cartridge or the spin chamber, and for reviewing dry powder levels capsules following inhalation. One or more cameras can be trained on the cartridge, the spin chamber, or other parts of the system that house capsules. In certain embodiments, an image processing system, bar code reader or RFID chip is associated with each capsule and/or the cartridge, and is used to identify that a capsule has been loaded, or otherwise used to verify the contents of the loaded dose.

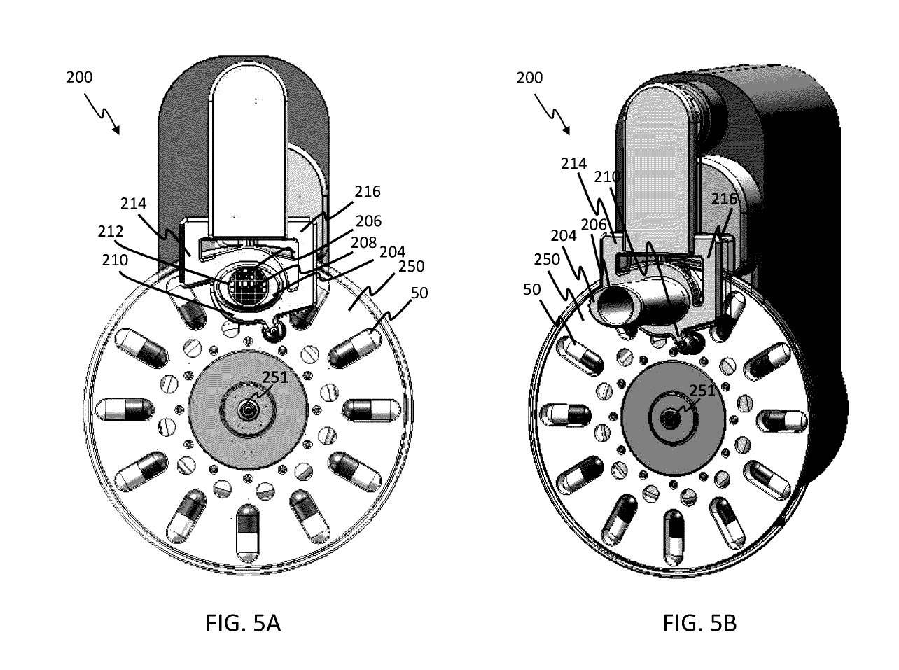

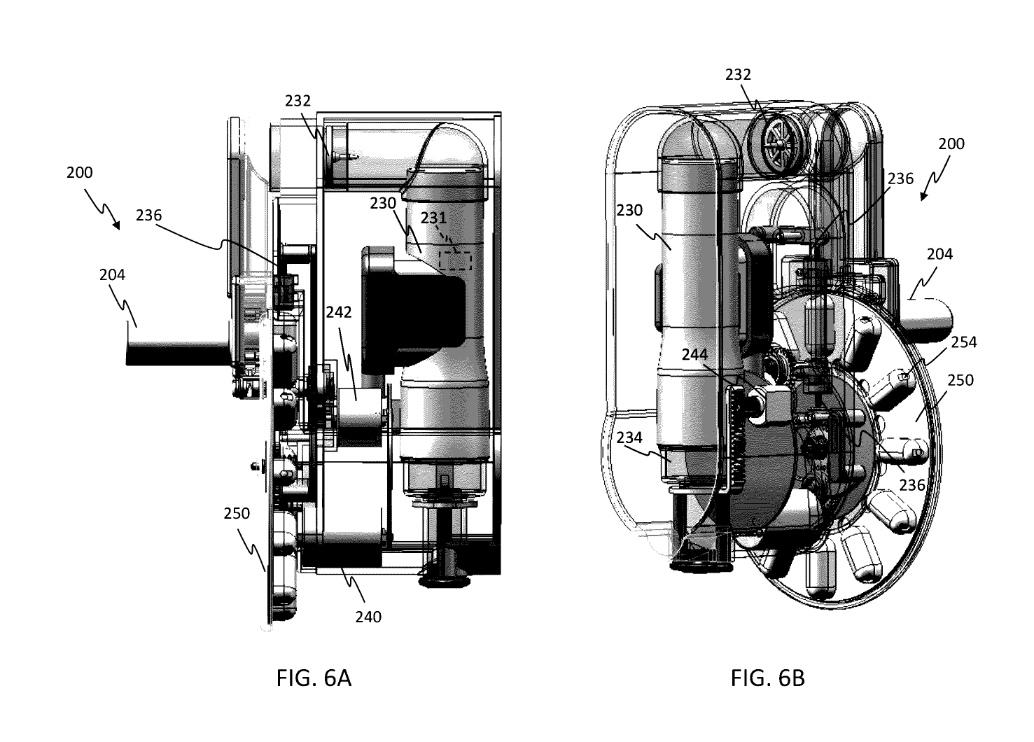

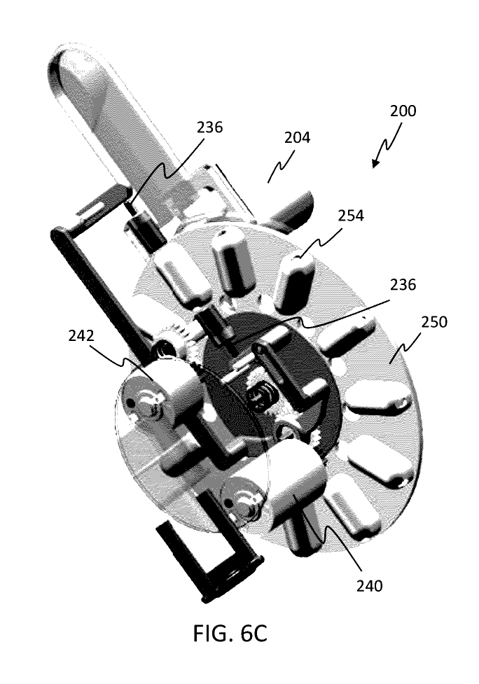

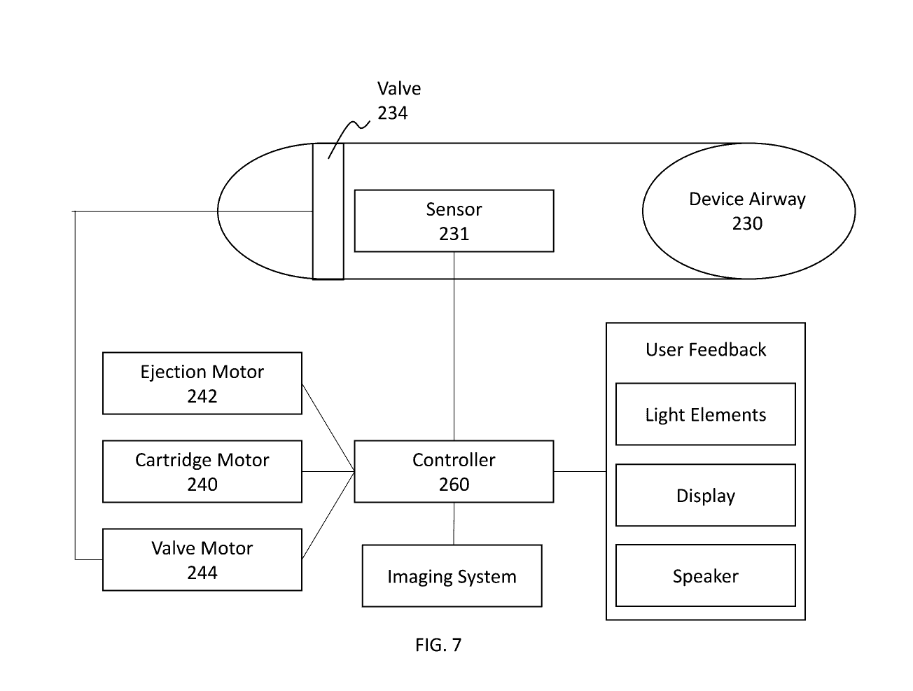

With reference now to one exemplary embodiment shown in FIGS. 5A-6B, a dry powder delivery system 200 is designed to accommodate a spinning cartridge 250. As shown in FIGS. 5A and 5B, the cartridge 250 spins about an axis 251 for advancing capsules, aligning capsules with the capsule port, and drawing capsules into the spin chamber 212. Here, capsules sit in receptacles spread radially about the axis on the cartridge 250 and are drawn into the spin chamber 212 using techniques similar to those described above as the receptacle holding the capsule is aligned with the capsule port. Used capsules drop from the spin chamber 212 when the ejection door 210 opens. The circular cartridge 250 holds multiple dry powder capsules 50 that may vary in content and quantity, similar to the previous embodiment. A mouthpiece 204 having a mouthpiece opening 206 forms an airway extending to the spin chamber 212. A lattice structure 208 is positioned in front of the spin chamber 212 in certain embodiments. First and second air inlets 214, 216 are connected to the spin chamber 212 in an offset configuration so that a negative air pressure generated at the mouthpiece opening 206 promotes a vortical flow in the spin chamber 212, facilitating drawing a capsule into the spin chamber and spinning of a loaded capsule. With reference now to FIGS. 6A-6C, electromechanical mechanisms and internal valve structures are shown according to an exemplary embodiment. A flow path 230 includes a flow or pressure sensor 231, such as a transducer. In certain embodiments, a one way valve 232, such as an elastomeric leaf or duckbill valve, is positioned in the airflow pathway, and configured to allow only a proximally directed inspiratory airflow towards the mouthpiece 204. A capsule puncturing pin (or pins) 236 is positioned next to the cartridge 250. In certain embodiments, the capsule puncturing pin 236 advances through a cartridge tray hole 254 to puncture one or more holes in a capsule 50 sitting within the tray. A first motor 240 is positioned behind the cartridge 250 to rotate the cartridge 250 about its axis 251. A second motor 242 is positioned behind the ejection door 210 for releasing capsules from the spin chamber. A third motor 244 opens and closes a valve 234 connected to the flow path 230. The valve 234 can open and close, acting as an inspiratory volume clamp. It is understood that gearing and mechanical elements can be arranged so that single motors and affect multiple functions ascribed to individual motors. Electromechanical elements such as the first 240, second 242 and third 244 motor, and other elements including the sensor 231, an imaging system and user feedback devices are operably connected to a controller 260, as shown in FIG. 7. The imaging system may be, for example, an arrangement of one or more cameras that can also be utilized to monitor the capsules and automate aspects of the challenge, as described herein.

The volume clamping mechanism of the device is able to address the limitations of other dry powder delivery systems for provocation testing. In order to get dry powders to flow into the airway, it requires that the test subject inhale with a sufficiently high flow that will pull the capsule into the spin chamber and create the vortices that spin the capsule. This flow may be as high as 30 or 120 liters of air per minute. However, if a subject is allowed to inhale at that flow rate without any volume limitation, they may quickly fill their lungs to capacity in less than a few seconds. As previously stated, this filling the lungs to capacity could blunt the response to the challenge of the particles. Therefore there is needed a volume clamping mechanism that allows the test subject to inhale with flow rates greater than 30 liters per minute and yet limits how much air they can inhale in terms of total volume. In one embodiment, the volume limit is set in the control logic of the device. During inhalation through flow sensor 231, the flow is integrated into its volume and when the volume reaches the set limit, valve 234 is closed and no additional air can enter the airway. The timing for the closure of the valve may be adjusted for the anticipatory time so that it is closed at the correct volume, taking into account the time to close. In certain embodiments, the limiting mechanism is configured to limit flow between 60 and 90 liters of air per minute.

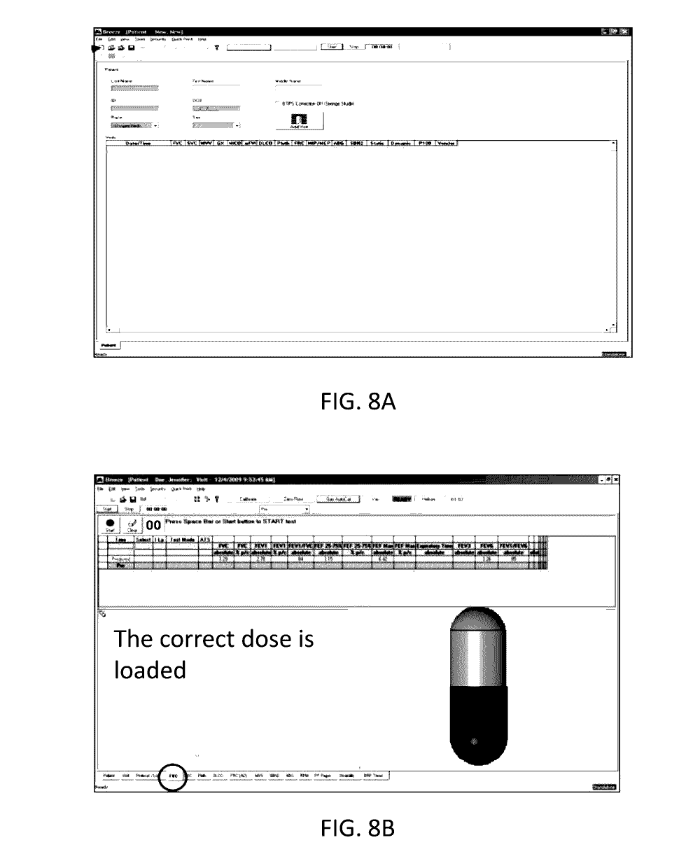

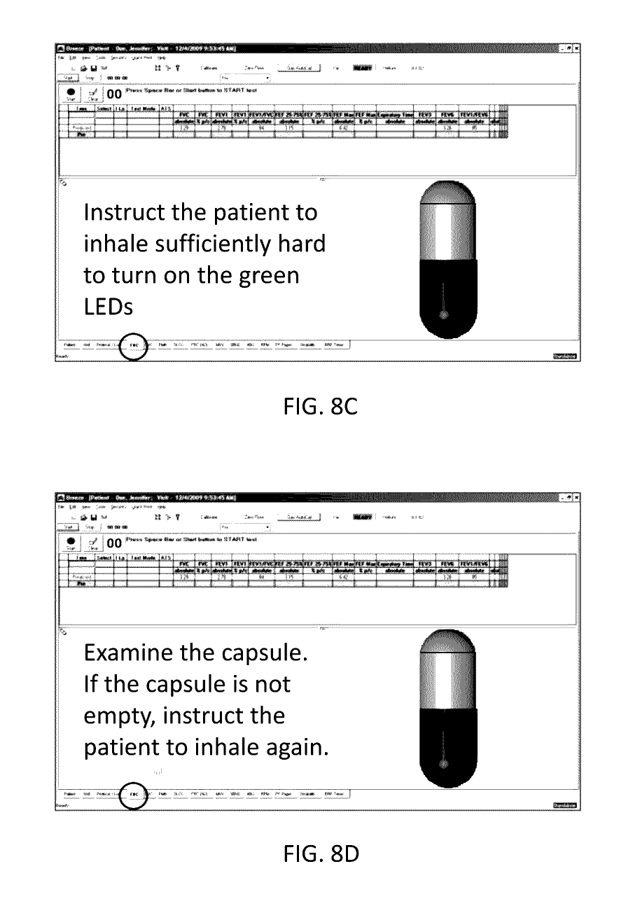

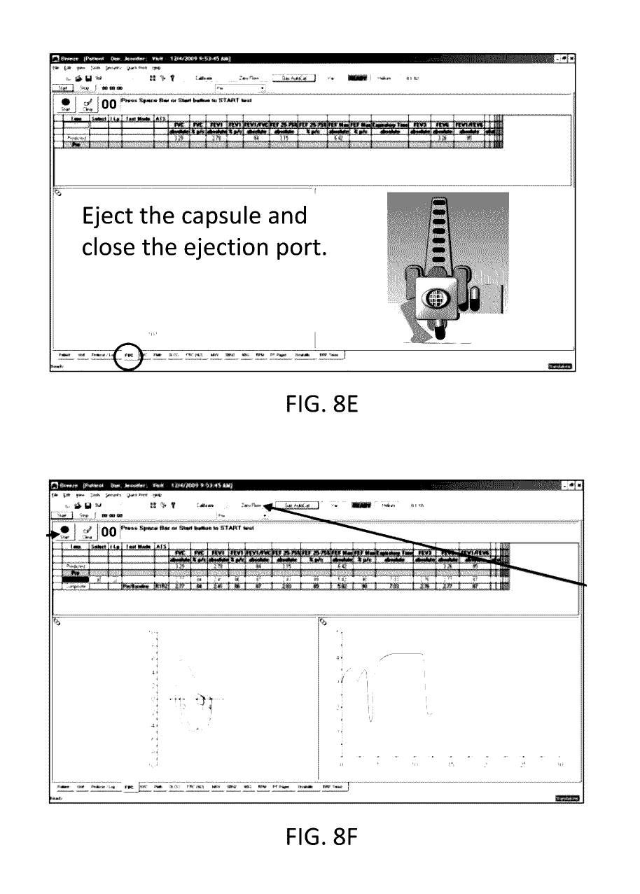

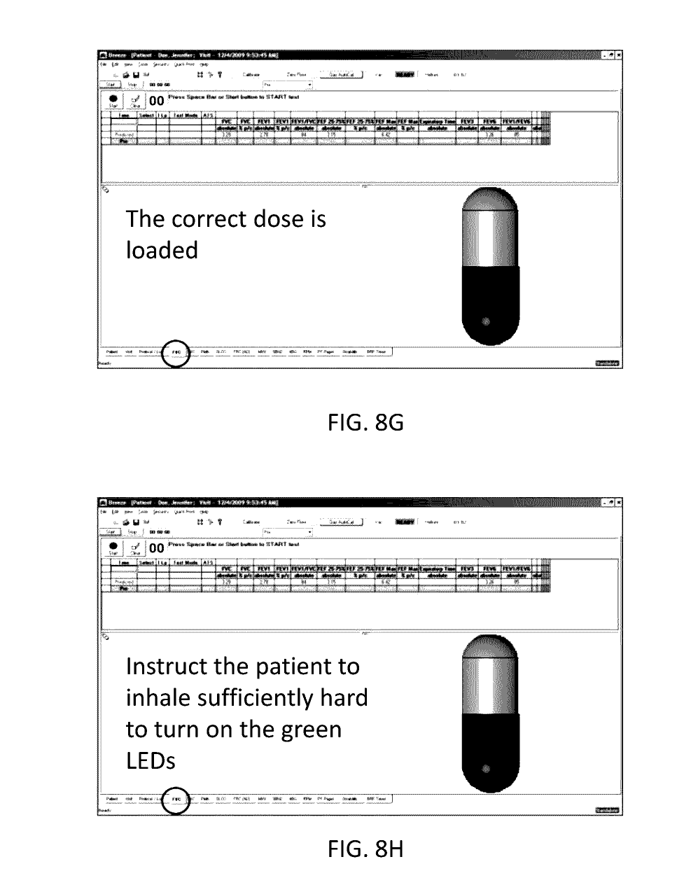

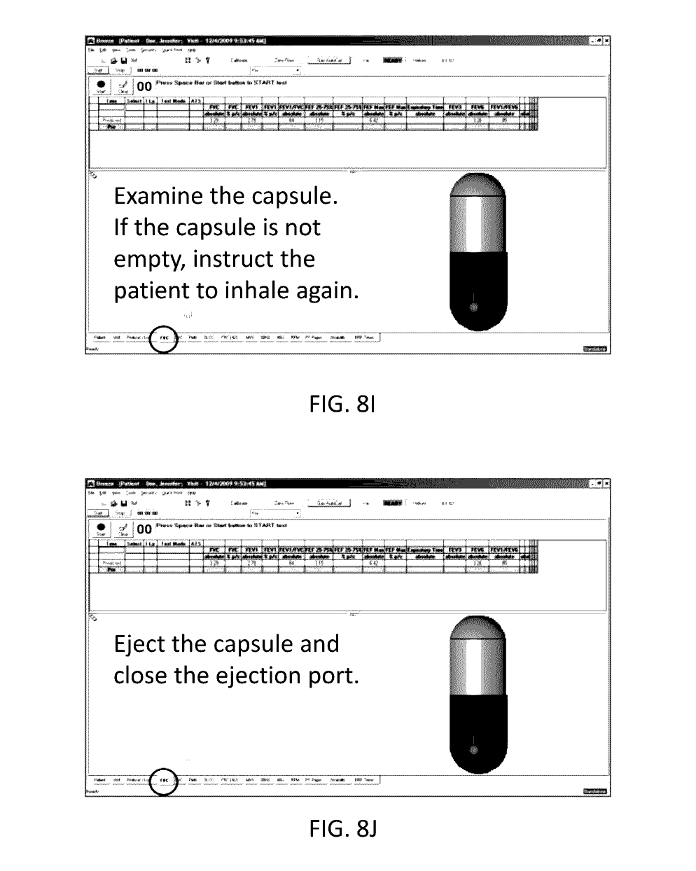

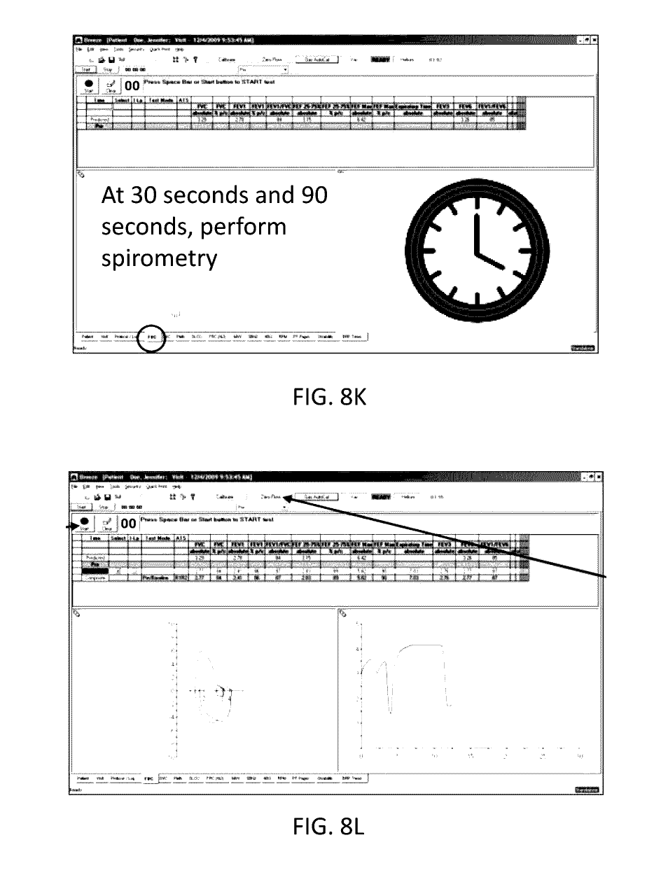

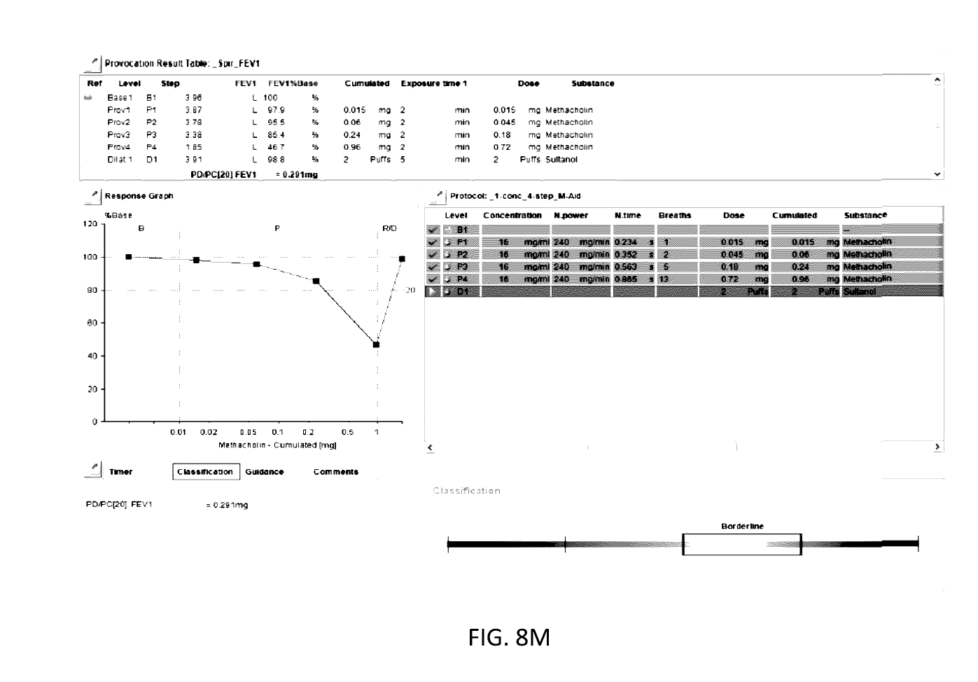

As mentioned above, the inhalation diagnostic challenge can include a number of visual and audio feedback mechanisms for the user and/or for the medical professional assisting the user in the challenge. Now with reference to FIGS. 8A-8M, an exemplary embodiment of a method for conducting a challenge procedure and an accompanying graphical user interface is shown. The graphical user interface can be displayed on any type of monitor or handheld mobile device, such as a tablet or a smart phone. An instructional tutorial can be loaded onto the system, giving the user a visual walkthrough of the procedure, so that they become familiar with what will be expected for a successful challenge. Instructions and results can also be accompanied by an audio readout from a connected speaker. Like many steps in this procedure, verification of dry powder clearance from the capsules can be done manually by a visual inspection from the technician or user. Further, actions such as advancing capsule cartridges, piercing capsules, and removing capsules from the spin chamber can likewise be performed automatically by elements such as motors and actuators, or by manual manipulation from a user or medical technician. At the beginning of the challenge procedure, user information is entered into the software (FIG. 8A). The software can be loaded onto a local memory unit coupled to the controller, or can be access remotely on a remote server. User input can be entered into the software using standard methods, such as keyboard input or by use of voice recognition software. In one embodiment, the first capsule is loaded into the spin chamber, and a camera imaging system checks the content and quantity of the capsule, and verifies that the correct dose is loaded (FIG. 8B). At this point and throughout the procedure, other sensors placed in the system can verify the status of various system components, such as whether or not the ejection door is properly closed, and whether or not certain valves and airways are open or shut. If a proper system state not verified at a given point during the procedure, an error message will appear on the screen with instructions on how to troubleshoot the problem, or possibly to abort the procedure. Once the system verifies that the correct dose is loaded, the capsule is punctured using one or more pins, and the user is instructed to inhale (FIG. 8C). Since the user inhalation has to be sufficiently hard to accurately perform the challenge, feedback elements such as light emitting diodes can light, indicating at a particular level whether or not the inhalation is strong enough. The user's inhalation is measured from an internal sensor. For example, red lights can indicate that an inhalation is insufficient, and green lights can indicate that an inhalation is at a sufficient level. Another visual feedback cue comes in the form of a spinning virtual capsule on the screen. After user inhalation is complete, the loaded capsule is examined to determine whether or not a sufficient amount of dry powder has been cleared from the capsule (FIG. 8D). As mentioned above, checking the capsule can be in the form of the technician giving it a visual inspection, or it can be done automatically by the imaging system. If the capsule is not sufficiently empty, the user will be instructed to take another strong inhalation. If the capsule is sufficiently empty, the capsule will be manually or automatically ejected from the spin chamber by opening the ejection door and removing it through an ejection port (FIG. 8E).

After the baseline forced vital capacity (FVC) is measured, the system will check for exclusion criteria (FIG. 8F). If it is determined that it is clear to proceed, the next instruction is to load the next capsule into the spin chamber. The system checks that the next dose is correct and properly loaded (FIG. 8G), the capsule is punctured, and the user is again instructed to inhale sufficiently hard while paying attention to user feedback cues (FIG. 8H). The technician or the imaging system then checks for clearance of the dry powder from the capsule (FIG. 8I). If the capsule is not sufficiently empty, the user is instructed to inhale again. Processing of inhalation performance by the system may generate some specific cues or types of encouragement for the user to increase their performance during the next inhale. If the powder is sufficiently cleared, the capsule is ejected (FIG. 8J), and the system moves on to instructions regarding FVC maneuvers. The user is instructed to perform an FVC maneuver at one or more points in time. In this exemplary embodiment, the user is instructed to perform the FVC maneuver at 30 seconds and 90 seconds (FIG. 8K). The timing can be tracked by a graphic that keeps track of time, such as a graphical clock or digital readout. In certain embodiment, after the first FVC maneuver, the system will display a graphical representation of user performance of the FVC maneuver (FIG. 8L). In certain embodiments, after the FVC maneuver, the system will calculate PD-20 and determine if the user should be tested at the next level of challenge (FIG. 8M). If the user is clear to proceed, an instruction will be given to load the next capsule. The system may also be designed to dynamically change the dosage based on real time feedback, so that dosages are strategically taken out of sequence. This can be based on a number of factors, such as physical patient characteristics, patient habits and medical history, results from baseline tests, and real time results and adjustments based on particular inhalation valves during the challenge. Further, a suggestion can be made to a medical professional for a next dosage, and the medical professional can either accept or override the suggestion based on their professional assessment.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A dry powder delivery device comprising:
a mouthpiece comprising a proximal and distal opening with a first passageway extending therebetween;
a spin chamber comprising a port and an ejection door, wherein the spin chamber is connected to the first air passageway;
a housing comprising a second passageway, wherein the second passageway is connected to the first passageway and further comprises an airflow sensor;
a controller operably connected to the airflow sensor; and
a cartridge comprising a plurality of dry powder receptacles that are configured to align with the port as the cartridge moves relative to the housing.

2. The dry powder delivery device of claim 1, wherein the second passageway comprises an adjustable limiting mechanism configured to limit inhaled volume through the second passageway.

3. The dry powder delivery device of claim 2, wherein the adjustable limiting mechanism is configured to limit inhaled volume through the second passageway to a level between 0.1 to 3.0 liters of air.

4. The dry powder delivery device of claim 2, wherein the adjustable limiting mechanism is configured to limit flow through the second passageway to a level between 30 and 120 liters of air per minute.

5. The dry powder delivery device of claim 2, wherein the adjustable limiting mechanism is controlled by a motor operably connected to the controller.

6. The dry powder delivery device of claim 4, wherein the adjustable limiting mechanism is a valve.

7. The dry powder delivery device of claim 1, wherein the cartridge is configured to move through the housing in a substantially linear direction.

8. The dry powder delivery device of claim 1, wherein the cartridge is configured to move through the housing in a substantially radial direction.

9. The dry powder delivery device of claim 1, wherein the cartridge is moved through the housing by a motor operably connected to the controller.

10. The dry powder delivery device of claim 1, wherein the cartridge is substantially rectangular.

11. The dry powder delivery device of claim 1, wherein the cartridge is substantially circular.

12. The dry powder delivery device of claim 1, wherein the sensor is a pressure transducer.

13. The dry powder delivery device of claim 1, wherein the mouthpiece is a detachable mouthpiece connected to the housing.

14. The dry powder delivery device of claim 1, wherein a lattice structure is disposed across at least one of the distal opening and the spin chamber.

15. The dry powder delivery device of claim 1, wherein a first and second air inlet are connected to the spin chamber and positioned to facilitate vortical airflow when a negative pressure is applied to the mouthpiece.

16. The dry powder delivery device of claim 1, wherein the housing comprises a performance indicator comprising a plurality of light emitting elements.

17. The dry powder delivery device of claim 16, wherein the plurality of light emitting elements comprises a first and second light emitting elements, each having different colors.

18. The dry powder delivery device of claim 1, wherein the ejection door is actuated by a motor operably connected to the controller.

19. The dry powder delivery device of claim 1 further comprising a piercing element configured to advance at least partially into the spin chamber.

20. The dry powder delivery device of claim 1 further comprising an imaging system comprising a first camera trained on the spin chamber and operably connected to the controller.

21. The dry powder delivery device of claim 1, wherein the imaging system comprises a second camera trained on the cartridge and operably connected to the controller.

22. The dry powder delivery device of claim 1, wherein the second pathway comprises a one-way elastomeric valve configured to favor proximally directed inspiratory airflow towards the mouthpiece.

23. A method for performing an inhalation diagnostic challenge comprising:
advancing a cartridge comprising a plurality of dry powder capsules through a device housing to deposit a first dry powder capsule into a spin chamber;
inhaling on a mouthpiece of the device to generate a vortical airflow within the spin chamber to spin the first capsule, the first capsule containing dry powder containing a first amount of methacholine;
releasing at least a first portion of the dry powder from the first capsule; and
inhaling an aerosolized form of the first portion of the dry powder released from the capsule.

24. The method of claim 23 further comprising:
advancing the cartridge in a linear direction through the device.

25. The method of claim 23 further comprising:
advancing the cartridge in a radial direction through the device.

26. The method of claim 23 further comprising:
adjusting an airflow through the device by adjusting an volume limiting mechanism.

27. The method of claim 23 further comprising:
ejecting the capsule from the spin chamber.

28. The method of claim 27 further comprising:
advancing the cartridge through the device to deposit a second dry powder capsule into the spin chamber;
inhaling on the mouthpiece of the device to generate a vortical airflow within the spin chamber to spin the second capsule, the second capsule containing dry powder containing a second amount of methacholine different from the first amount of methacholine;
releasing at least a second portion of the dry powder from the second capsule; and
inhaling an aerosolized form of the second portion of the dry powder released from the capsule.

29. The method of claim 28, wherein the second amount of methacholine is greater than the first amount of methacholine.

30. The method of claim 29, wherein the second amount of methacholine is substantially two times greater than the first amount of methacholine.

31. The method of claim 29, wherein the second amount of methacholine is substantially four times greater than the first amount of methacholine.

32. A dry powder delivery device comprising:
a mouthpiece comprising a proximal and distal opening with a first passageway extending therebetween;
a spin chamber comprising a port and an ejection door, wherein the spin chamber is connected to the first air passageway;
a housing comprising a second passageway, wherein the second passageway is connected to the first passageway and further comprises an airflow sensor and an adjustable limiting mechanism configured to limit inhaled volume through the second passageway; and
a controller operably connected to the airflow sensor and the adjustable limiting mechanism.

33. The dry powder delivery device of claim 32, wherein the adjustable limiting mechanism is configured to limit inhaled volume through the second passageway to a level between 0.1 to 3.0 liters of air.

34. The dry powder delivery device of claim 32, wherein the adjustable limiting mechanism is configured to limit flow through the second passageway to a level between 30 and 120 liters of air per minute.

35. The dry powder delivery device of claim 32, wherein the adjustable limiting mechanism is controlled by a motor operably connected to the controller.

36. The dry powder delivery device of claim 32, wherein the adjustable limiting mechanism is a valve.

37. The dry powder delivery device of claim 32 further comprising:
a cartridge comprising a plurality of dry powder receptacles that are configured to align with the port as the cartridge moves relative to the housing.

38. A method for performing an inhalation diagnostic challenge comprising:
advancing a cartridge comprising a plurality of dry powder receptacles through a device housing to deposit a first amount of dry powder into a spin chamber;
inhaling on a mouthpiece of the device to generate a vortical airflow within the spin chamber, the first amount of dry powder containing a first amount of methacholine; and
inhaling an aerosolized form of the first amount of dry powder.

39. The method of claim 38 further comprising:
advancing the cartridge through the device to deposit a second amount dry powder into the spin chamber;
inhaling on the mouthpiece of the device to generate a vortical airflow within the spin chamber, the second amount of dry powder containing a second amount of methacholine different from the first amount of methacholine; and
inhaling an aerosolized form of the second amount of dry powder.

40. The method of claim 39, wherein the second amount of methacholine is greater than the first amount of methacholine.

41. The method of claim 40, wherein the second amount of methacholine is substantially two times greater than the first amount of methacholine.

42. The method of claim 40, wherein the second amount of methacholine is substantially four times greater than the first amount of methacholine.
